(19) United States
(12) Patent Application Publication
(10) Pub. No.: US 2023/0346109 A1
(43) Pub. Date: Nov. 2, 2023

(54) SYSTEM FOR PERSONALIZED SKIN TREATMENT INCLUDING DISPENSER AND CARTRIDGE-BASED APPLICATOR

(71) Applicant: L'Oreal, Paris (FR)

(72) Inventors: Gregoire CHARRAUD, Jersey City, NJ (US); Casey BARBARINO, San Anselmo, CA (US); Rafael FELICIANO, New Providence, NJ (US)

(73) Assignee: L'Oreal, Paris (FR)

(21) Appl. No.: 17/731,984

(22) Filed: Apr. 28, 2022

(51) Int. Cl. A45D 40/26; A61N 5/06; A45D 44/00; G16H 10/20

(52) U.S. Cl. CPC A45D 40/261; A61N 5/0616; A45D 44/005; G16H 10/20; A61N 2005/0663; A45D 2200/058; A45D 2200/205; A45D 2044/007

(57) ABSTRACT

A system for dispensing a skin formula, the system including a base device configured to dispense a formula into an applicator, an applicator configured to apply the formula, the applicator including a reservoir configured to hold the formula, a roller ball configured to apply the formula, and an attachment configured to attach the applicator to the base device and a dispensing device.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a system for dispensing a skin formula, the system including a base device configured to dispense a formula into an applicator, an applicator configured to apply the formula, the applicator including a reservoir configured to hold the formula, a roller ball configured to apply the formula, and an attachment configured to attach the applicator to the base device and a dispensing device is disclosed.

In another aspect, a method of using the skin care system according to claim 1, wherein the method includes placing an applicator into a base device; filling the applicator with a formula, removing the applicator from the base device, placing the applicator into a dispensing device, and applying the formula with the applicator while simultaneously administering light therapy with the dispensing device is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an example applicator, in accordance with the present technology;

FIG. 2A-2B are example cross-sections of an applicator, in accordance with the present technology;

FIG. 3A-3B are example base device, in accordance with the present technology;

FIG. 4 is an example dispensing device, in accordance with the present technology;

FIG. 5 is an example skin care system, in accordance with the present technology; and

FIG. 6 is an example method of using a skin care system, in accordance with the present technology.

### DETAILED DESCRIPTION

While illustrative embodiments have been illustrated and described, it will be appreciated that various changes can be made therein without departing from the spirit and scope of the invention.

Described herein is a system for filling and cleaning an applicator configured to apply a formula in conjunction with a dispensing device. A base device is configured to fill and clean an applicator and allow a user to mix one or more formula ingredients into a formula for skin care. In some embodiments, after filling the applicator, a user can attach the applicator to a dispensing device configured to administer light treatment while the applicator applies a formula to a surface, such as skin. In some embodiments, a connected communication device can allow a user to select a formula comprised of one or more formula ingredients. In some embodiments, the formula may be a moisturizer, a concealer, a wrinkle or fine line treatment, a toner, or an acne treatment. In some embodiments, the base device includes one or more cartridges containing a formula ingredient, including but not limited to aloe, vitamin C, coconut milk, or shea butter. When a user selects a desired formula with an application on the communication device, one or more formula ingredients can be dispensed into the applicator to create the personalized formula.

In some embodiments, the user can apply the formula to their skin while light therapy, such as NIR light therapy, red light therapy, blue light therapy, or yellow light therapy to further treat the skin.

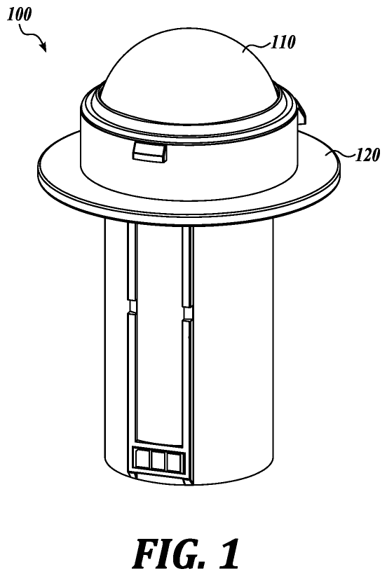

FIG. 1 is an example applicator, in accordance with the present technology. The applicator 100 may include a roller ball 110, and an attachment 120.

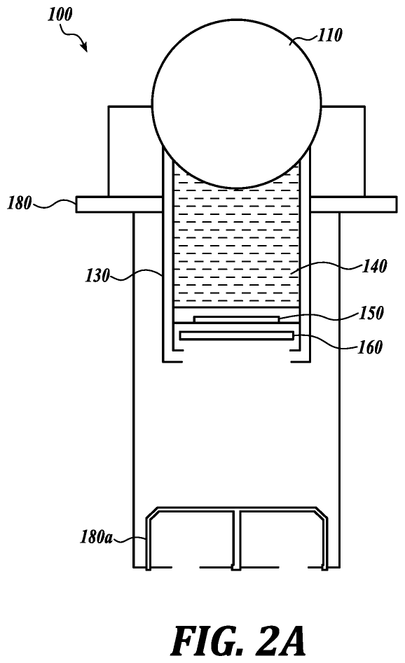

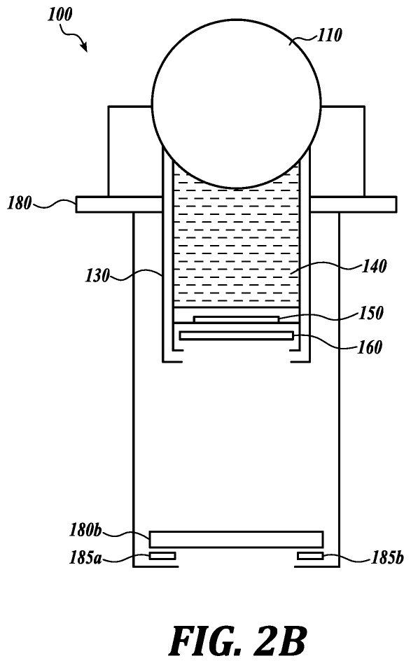

The roller ball 110 may be configured to distribute and apply a formula located a reservoir inside the applicator 100 (as shown in FIGS. 2A-2B). In some embodiments, the roller ball 110 is plastic, but in other embodiments, the roller ball 110 may be glass or metal.

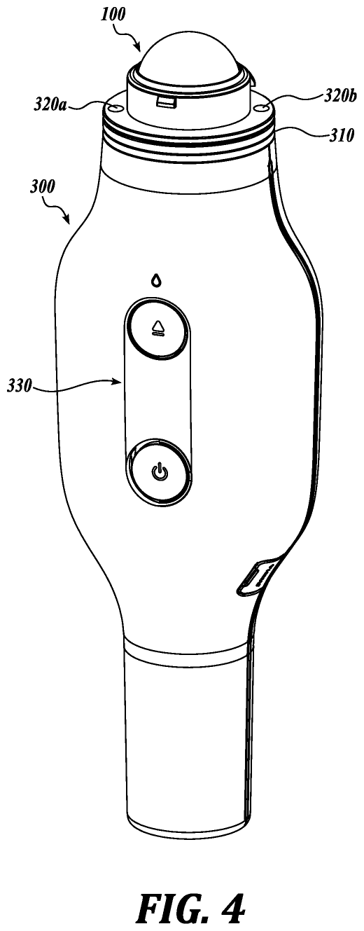

In some embodiments, the applicator 100 also includes an attachment 120 configured to secure the applicator 100 into a dispensing device, such as the dispensing device 200 in FIG. 4. While the attachment 120 is illustrated as a disk shaped to couple to a dispensing device, the attachment 120 may take any form capable of securing the applicator to a dispensing device including a threaded attachment, a magnet, or an attachment configured to snap into the dispensing device. In some embodiments, the attachment 120 is clear so that the dispensing device is visible through the attachment.

In operation, the applicator 100 can be placed inside a dispensing device (as shown in FIG. 4) and secured to the dispensing device with the attachment 120. The roller ball 110 can be rolled over a surface, such as a user's skin, to apply a formula.

FIG. 2A-2B are example cross-sections of an applicator, in accordance with the present technology. The applicator 100 may include a roller ball 110, an attachment 120, a reservoir 130 configured to hold a formula 140, a piston 150, a microcontroller 160, and a processor 170.

In some embodiments, the reservoir 130 is located inside the applicator 100, and is configured to hold a formula. In some embodiments, the formula is a skin care formula. In some embodiments, the skin care formula is a moisturizer, a toner, an acne treatment, a wrinkle treatment, fine line treatment, or a cosmetic. As the roller ball 110 rolls, formula 140 from the reservoir 130 is applied to a surface.

In some embodiments, the applicator 100 further includes a piston 150 configured to push the formula 140 towards the roller ball 110 as the formula is applied. In some embodiments, the piston 150 is directed by circuitry on a dispensing device or on the applicator itself to push the formula 140.

In some embodiments, the applicator 100 includes a microcontroller 160 configured to identify the type of formula 140 inside the applicator 100 to a dispensing device. The microcontroller 160 may be used to identify any number of things about the formula 140 or applicator 100, including the amount of formula 140 inside the applicator 100, the expiration date of the formula 140 inside the applicator 100, or when to replace the applicator.

FIG. 1A is a cross section of an example applicator 100 with an example filling mechanism 180a. In some embodiments, the applicator 100 includes a filling mechanism 180 configured to couple to a base device and fill the reservoir 130 of the applicator 100.

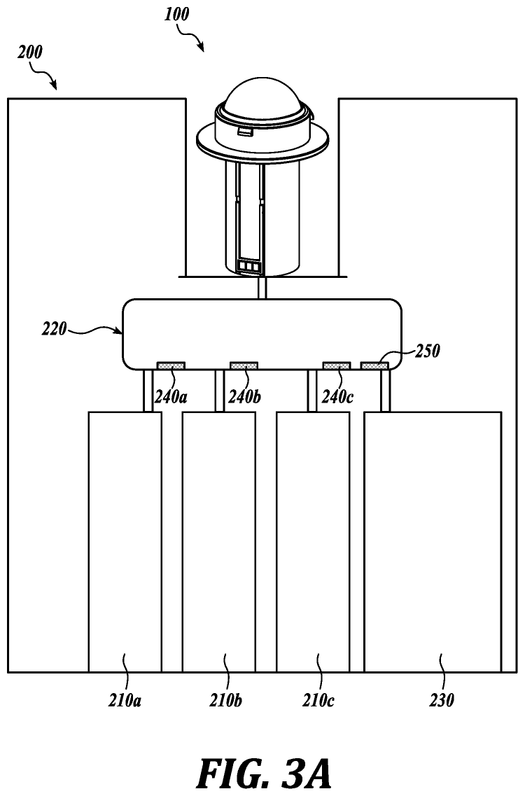

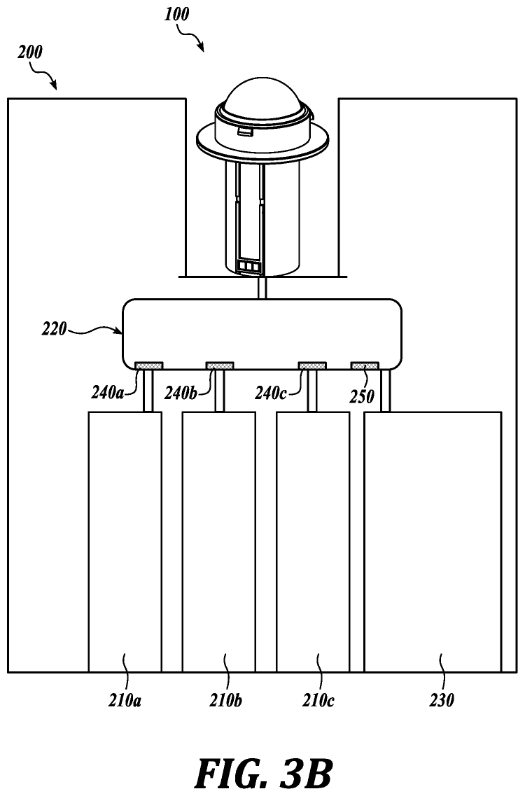

In some embodiments, the filling mechanism 180 is an umbrella valve 180a. In operation, when the applicator 100 is connected to a base device (such as shown in FIGS. 3A-3B), the umbrella valve 180 raises to allow formula 140 to flow from base device to the reservoir 130 of the applicator.

FIG. 1B is a cross section of an example applicator 100 with another example filling mechanism 180b. In some embodiments, the applicator 180 includes two or more magnets 185a, 185b and a magnetic filler 180b.

In operation, when the applicator 100 is placed on a base device, the two or more magnets 185a, 185b generate a magnetic field to raise the magnetic filler 180b. As the magnetic filler 180b is raised, formula 140 can dispense from the base device into the reservoir 130 of the applicator 100.

FIG. 3A-3B are example base devices, in accordance with the present technology. The base device 200 may include a dispensing chamber 220, one or more cartridges 210a, 210b, 210c, and a cleaning solution reservoir 230. In some embodiments, the base device 200 may also include one or more cartridge lids 240a, 240b, 240c and a cleaning solution lid 250.

In some embodiments, the one or more cartridges 210a, 210b, 210c have a round, cylindrical body and a nozzle at a first end. The cartridge 210 may contain and dispense an amount of a formula ingredient into the dispensing chamber 220 as needed. In some embodiments, the cartridge 210 dispenses a formula ingredient by rotation of a cartridge gear while the cartridge 210 remains in place substantially vertically along the Y-axis. A circumferential groove is at a second end of the cartridge and is used to secure the cartridge 210 into the dispensing device 100.

An amount of formula ingredient may be released from the cartridge 210 through the nozzle by a first rotational motion of the first end with respect to the second end of the cartridge 210. Rotational motion of the first end of the cartridge 210 in a second direction, opposite of the first rotational motion, may close the nozzle of the cartridge 210.

In operation, an applicator 100 may be placed onto the base device 200. In some embodiments, the one or more cartridges 210a, 210b, 210c dispense one or more formula ingredients into the mixing chamber 220. While the one or more cartridges 210a, 210b, 210c are dispensing the formula ingredients, as shown in FIG. 3A, the one or more cartridge lids 240a, 240b, 240c are configured to allow the formula ingredients to flow from the one or more cartridges 210a, 210b, 210c into the mixing chamber 220. In the mixing chamber, the one or more formula ingredients are mixed to produce a formula. In some embodiments, the formula may consist of a single formula ingredient. The mixing chamber than extrudes the formula into the applicator 100. As the applicator 100 is being filled with a formula (such as formula 140) the cleaning solution lid 250 is configured to block cleaning solution from entering the mixing chamber 220 and the applicator 100.

In some embodiments, the base device 200 further includes a cleaning reservoir 230 configured to hold a cleaning solution. In some embodiments, the cleaning reservoir is another cartridge 210a, 210b, 210c, but in other embodiments, the cleaning reservoir 230 may be larger than the cartridges. In some embodiments, the cleaning reservoir 230 may be removable from the base device 200.

The base device 200 may also be configured to clean the applicator between formulas. In operation, as shown in FIG. 3B, the cleaning reservoir 230 hold a cleaning solution. The cleaning solution reservoir 230 dispenses cleaning solution into the mixing chamber 220. The mixing chamber 230 can then extrude the cleaning solution into the applicator 100 to clean the applicator 100. When the cleaning solution reservoir 230 is dispensing cleaning solution into the mixing chamber 220, the cleaning solution lid is configured to allow cleaning solution to flow from the cleaning solution reservoir 230 into the mixing chamber. Further, the cartridge lids 240a, 240b, 240c are configured to prevent one or more formula ingredients from flowing into the mixing chamber 220.

FIG. 4 is an example dispensing device, in accordance with the present technology. In some embodiments, the applicator 100 can be attached to a dispensing device 300. In some embodiments, the dispensing device includes an end 310, one or more light sources 320a, 320b, and an actuator 330. In some embodiments, the applicator 100 connects to the dispensing device 300.

In some embodiments, the dispensing device 300 includes an end 310. The end 310 may be configured to be seen through the attachment 120 on the applicator 100. In some embodiments, the end 310 includes one or more light sources 320a, 320b configured to administer light treatment to a surface while the formula 140 is being applied.

In some embodiments, the one or more light sources 320a, 320b are LEDs. In some embodiments, there are only two light sources 320a, 320b on the dispensing device. In some embodiments, a first light source 320a is configured to administer light therapy in a first wavelength. In some embodiments, a second light source 320b is configured to administer light therapy in a second wavelength. In some embodiments, the light therapy in the first wavelength and the light therapy in the second wavelength are administered simultaneously. In some embodiments, the light therapy and applying the formula happen simultaneously.

In some embodiments, the dispensing device 300 includes one or more actuators 330. While the actuator is illustrated as a button, in some embodiments, the actuator may be a switch, a capacitive touch type button, a dial, or the like. The actuator may be configured to begin the administration of light therapy, to apply the formula, or both.

In some embodiments, the dispensing device 300 also includes a contact-less chip reader (not pictured in FIG. 2) to read the microcontroller 160 on the applicator 100.

In operation, a user may place an applicator 100 into the dispensing device 300. When the actuator 330 is actuated, the formula is applied, the light therapy is administered, or both, simultaneously. A user may then apply the formula with the applicator 100.

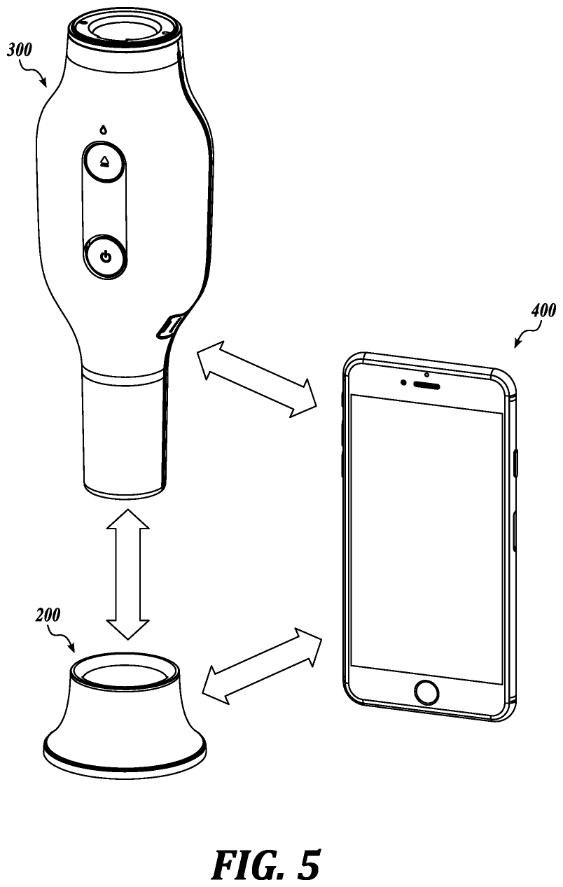

FIG. 5 is an example skin care system, in accordance with the present technology. The system, which implements the base device 200 described above, includes at least the hair base device 200, a dispensing device 300 as described herein, and a connected communication device 300. Optionally, the system may further include one or more external servers which are implemented as part of a cloud-computing environment.

The communication device 400 may be a personal computer (PC), a laptop computer, a PDA (Personal Digital Assistants), a smart phone, a tablet device, a UMPC (Ultra Mobile Personal Computer), a netbook, or a notebook type personal computer. In the below examples, the connected device 300 is assumed to be a smartphone, such as an Apple iPhone.

The communication device 400 is capable of performing wireless communication with the base device 200 and/or the dispensing device 300 by way of a wireless communication interface circuitry on the hair care dispenser 100. However, communication device 400 is also capable of having a wired connection to the base device 200 and/or dispensing device 300 by way of a USB interface on the apparatus 100. Additionally, each device, including the applicator 100, may communicate with each other and the external one or more devices through an internet connection via an 802.11 wireless connection to a wireless internet access point, or a physical connection to the internet access point, such as through an Ethernet interface. Each connected communication device 300 is capable of performing wireless communication with other devices, such as through a Bluetooth connection or other wireless means as well.

The connected communication device 300 is configured to receive information from a user for use in generating a hair care formulation that will be used by the base device 200 to dispense hair care material into a mixing chamber, such as mixing chamber 220.

In some embodiments, the communication device 400 has a related application on it, configured to aid a user in dispensing a formula and/or cleaning an applicator 100. In some embodiments, the application is configured to apply an algorithm to a photo or video of a user to detect one or more skin features. In some embodiments, the one or more skin features may include a dark circle, acne, pigmentation, rosacea, wrinkles, fine lines, or wounds. Additionally, in some embodiments, the application is configured to diagnose one or more skin conditions using an AI algorithm.

In some embodiments, the communication device 400 may further sense one or more environmental conditions with one or more sensors. In some embodiments, the environmental conditions may include temperature, humidity, UV, pollution, and the like. In some embodiments, the application can gather environmental data from other sources such as weather services. In some embodiments, the application can further recommend a formula, comprised of one or more skin ingredients, to the user based on the detected skin feature and/or environmental conditions.

In some embodiments, the user can set up a user profile on the application of the communication device 400. In some embodiments, setting up the user profile includes answering a user questionnaire. In some embodiments, the user questionnaire gives the user a series of inputs including past skin treatment, past use of the applicator, desired skin quality, or skin concern. In some embodiments, the application can solicit feedback from the user regarding their favorite or most effective formulation to help improve the algorithm.

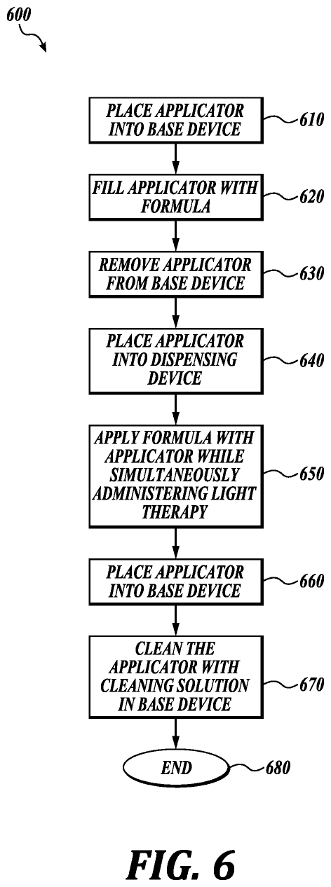

FIG. 6 is an example method 600 of using a skin care system, in accordance with the present technology.

In block 610, an applicator (such as applicator 100) is placed into a base device. In some embodiments, the applicator may slide into, or click into the base device. In some embodiments, the applicator may include an attachment for attaching to the base device, such as a clip or a magnet.

In block 620, the applicator is filled with formula. In some embodiments, the applicator is filled with formula as described in FIG. 3A. In some embodiments, one or more cartridges may be placed inside the base device to dispense one or more formula ingredients into a mixing chamber. In some embodiments, the mixing chamber can then extrude the formula into the applicator.

In block 630, the applicator is removed from the base device.

In block 640, the applicator is placed into the dispensing device. In some embodiments, the applicator may be placed into the dispensing device with the same attachment that connected the applicator to the base device. In some embodiments, the applicator 100 may be pre-filled. In that case, the method may start from block 640.

In block 650, formula is applied with the applicator while light therapy is being administered simultaneously. In some embodiments, the dispensing device includes an actuator configured to begin both the application of the formula and the administration of the light therapy. In some embodiments, the dispensing device includes two or more light sources configured to administer the light therapy. In some embodiments, the dispensing device is configured to deliver two or more types of light therapy, either one at a time, or simultaneously. In such embodiments, one light source is configured to administer a first light therapy, and another light source is configured to administer a second light therapy. In some embodiments, the first light therapy may include emitting a first light at a first wavelength, and the second light therapy may include emitting a second light at a second wavelength.

In block 660, the applicator is returned to the base device. In some embodiments, the applicator may be used more than once before the formula inside the applicator has been used up. In some embodiments, the applicator holds enough formula for only a single use.

In block 670, the applicator is cleaned with cleaning solution inside the base device, as described herein. In some embodiments, a user may repeat block 670 to thoroughly clean the applicator. In some embodiments, the applicator may then be filled up with a formula once again for another use.

In block 680, the method ends.

The embodiments of the invention in which an exclusive property or privilege is claimed are defined as follows:

1. A system for dispensing a skin formula, the system comprising:
a base device configured to dispense a formula into an applicator;
an applicator configured to apply the formula, the applicator comprising:
a reservoir configured to hold the formula,
a roller ball configured to apply the formula, and
an attachment configured to attach the applicator to the base device and a dispensing device.

2. The system of claim 1, wherein the base device comprises at least one cartridge containing a formula.

3. The system of claim 2, wherein the base device is further configured to dispense the formula from the at least one cartridge into the applicator.

4. The system of claim 1, wherein the base device further comprises a reservoir, configured to hold a cleaning solution.

5. The system of claim 4, wherein the base device is further configured to dispense a cleaning solution from the reservoir into the applicator.

6. The system of claim 2, wherein the base device further comprises at least two cartridges, wherein each cartridge is configured to hold a formula ingredient.

7. The system of claim 6, wherein the base device is configured to dispense one or more formula ingredients into the applicator, so that the one or more formula ingredients mix inside the applicator to make a formula.

8. The system of claim 1, wherein the system further comprises a communication device.

9. The system of claim 8, wherein the communication device is a smartphone.

10. The system of claim 1, wherein the system further comprises a dispensing device configured to be coupled to the applicator with the attachment on the applicator, and wherein the dispensing device is configured to apply the formula and administer a light therapy simultaneously.

11. A method of using the skin care system according to claim 1, wherein the method comprises:
placing an applicator into a base device;
filling the applicator with a formula;
removing the applicator from the base device;
placing the applicator into a dispensing device; and
applying the formula with the applicator while simultaneously administering light therapy with the dispensing device.

12. The method of claim 11, wherein the method further comprises cleaning the applicator with a cleaning solution dispensed from the base device.

13. The method of claim 11, wherein the method further comprises:
selecting one or more skin ingredients on an application on a communication device;
dispensing the one or more skin ingredients from one or more cartridges on the base device to create the formula; and
filling the applicator with the formula.

14. The method of claim 11, wherein method further comprises:
administering light therapy with the dispensing device, wherein the light therapy comprises emitting two or more wavelengths of light simultaneously.

15. The method of claim 11, wherein the method further comprises identifying the formula in the applicator with the dispensing device.

16. The method of claim 11, wherein the method further comprises:
taking a photo of a user's skin with the communication device;
uploading the photo into an application on the communication device;
applying an algorithm to the photo to detect one or more skin features;
sensing one or more environmental conditions with one or more sensors;
recommending the formula based on the one or more skin features, and the one or more environmental conditions; and
dispensing the formula from the base device based into the applicator.

17. The method of claim 11, wherein the method further comprises setting up a user profile with the communication device.

18. The method of claim 17, wherein setting up the user's profile comprises answering a user questionnaire, wherein the user questionnaire gives the user a series of inputs, and wherein the series of inputs include past skin treatment, desired skin quality, or skin concern.

19. The method of claim 17, wherein the method further comprises diagnosing one or more skin conditions with an AI algorithm.

20. The method of claim 19, wherein the method further comprises accepting user feedback of the user's favorite formulations to improve the algorithm.
